(19) United States
(12) Patent Application Publication
(10) Pub. No.: US 2008/0033541 A1
(43) Pub. Date: Feb. 7, 2008

(54) Artificial mitral valve

(76) Inventors: Daniel Gelbart, Vancouver (CA); Samuel Victor Lichtenstein, Vancouver (CA)

Correspondence Address: Dan Gelbart, 4706 Drummond Dr., Vancouver, BC, V6T-1B4, US

(21) Appl. No.: 11/497,306

(22) Filed: Aug. 2, 2006

(51) Int. Cl. A61F 2/24

(52) U.S. Cl. 623/2.11; 623/2.18

(57) ABSTRACT

An artificial mitral valve is made of a short piece of elastomeric tubing having a one round end and one flattened end. The tubing can be rolled up to a small diameter and fits snuggly into the mitral valve opening when expanded. The tubing attaches to a few rings made of thin flexible wire. When the rings are expanded inside the left atrium, they form a support structure holding the artificial valve in the correct position. The rings can be flattened and delivered via a catheter together with the valve. The artificial valve contains no rigid component, therefore it does not deform or damage the area around the defective mitral valve and can be installed even in highly calcified or deteriorated valves.

### FIELD OF THE INVENTION

The invention relates to cardiac surgery, and in particular to percutaneous replacement of the mitral valve

### BACKGROUND OF THE INVENTION

Mitral valve degradation, such as regurgitation or stenosis, is a common problem affecting millions of people. In initial stages the problem is caused by imperfect sealing of the leaflets. This can be remedied by deforming the valve annulus to bring leaflet closer together, for better contact, or installing a device between the two leaflets in order to reduce the distance each leaflets needs to cover. Often those and other measure are insufficient and an artificial valve is required. There are many designs of prior art valves but a common problem is the anchoring of the artificial valve in a percutaneous procedure, where anchoring by suturing is not practical. U.S. patent application 2006/0058871 discloses a novel way of anchoring a pocket to reduce the distance between the valve leaflets. It was found that a similar method can also be used to support an artificial valve, which is no more complicated than the pocket used in the abovementioned patent but offers a more radical solution, even for non-operational valves. The anchoring method is combined with a novel valve design capable of being rolled up to fit through a moderate sized catheter and installed percutaneously. The area surrounding the mitral valve does not offer a natural ledge or annulus for anchoring an artificial valve. Because of the proximity to the aortic valve it is important than any artificial valve will be sufficiently soft not to distort and interfere with the aortic valve. The mitral valve is the most demanding cardiac valve also because it has to seal against the highest back pressure (up to 200 mmHg) of all other cardiac valves. The present invention offers a simple and reliable valve having no rigid parts, capable of being delivered via a catheter and being able to closely emulate a natural mitral valve.

### SUMMARY OF THE INVENTION

An artificial mitral valve is made of a short piece of elastomeric tubing having a one round end and one flattened end. The tubing can be rolled up to a small diameter and fits snuggly into the mitral valve opening when expanded. The tubing is attaches to a few rings made of thin flexible wire. When the rings are expanded inside the left atrium, they form a support structure holding the artificial valve in the correct position. The rings can be flattened and delivered via a catheter together with the valve. The artificial valve contains no rigid component, therefore it does not deform or damage the area around the defective mitral valve and can be installed even in highly calcified or deteriorated valves.

### BRIEF DESCRIPTION OF THE DRAWINGS

FIG. 1 is a cross section of the upper part of the heart, showing the artificial valve being delivered via a catheter.

FIG. 2 is a cross section showing the deployed valve.

FIG. 3 is an isometric view of the valve and support structure.

FIG. 4 is an isometric view of the valve folded inside the delivery catheter.

FIG. 5 is a cross section of the valve in the closed position.

FIG. 6 is a cross section of the valve in the open position.

FIG. 7 shows different cross section of the valve.

### DESCRIPTION OF THE PREFERRED EMBODIMENT

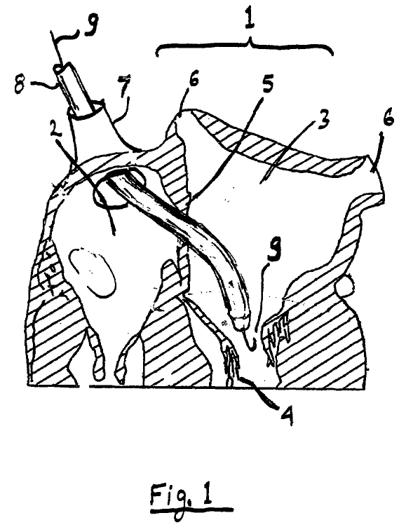

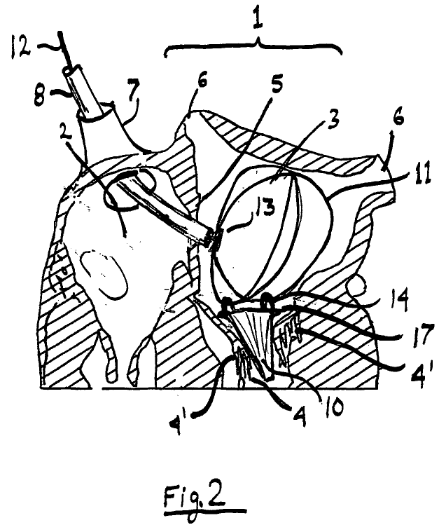

Referring now to FIG. 1, a catheter 9 is inserted into the left atrium 3 of heart 1 in order to reach defective mitral valve 4. Catheter 8 can be inserted through septum 5 via the vena cava 7 and right atrium 2, via the pulmonary veins 6 or by using any of the methods known in the art for minimally invasive or percutaneous cardiac surgery. Typically catheter 8 is guided by guide wire 9. The art of inserting catheters into the mitral valve is well known. Once tip of catheter 8 reaches mitral valve 4 the artificial valve is pushed out and catheter is retracted as shown in FIG. 2.

Referring now to FIG. 2, the flexible artificial valve 10 fits snuggly in the opening of mitral valve 4. Mitral valve leaflets 4′ help with forming a hemostatic seal as ventricular blood pressure pushes them against artificial valve 10. Even if leaflets 4′ separate from valve 10 during diastolic phase the form a secondary valve around artificial valve 10. Expanded wire rings 11 are supported by the walls of atrium 3 and prevent valve 10 from being pushed back into atrium 3 by the ventricular back pressure. Assuming a valve area of 3 sq.cm. and ventricular pressure of 125 mmHg, the force on the closed valve is about 500 gm, which requires wires 11 not to buckle under this force. This is achieved by using multiple wire rings. Other support structures can be used, such as mesh-shaped structure made of metal or flexible polymer. Wires 11 are temporarily attached to flexible cable 12 by coupler 13, which could be simply a female thread on 13 and a male thread at the tip of cable 12. After cable 12 is pushed in to fully expand wires 11, it is detached from coupler 13. Up to this stage the operation is fully reversible and the artificial valve can be pulled back into catheter 8. This allows fully testing valve 10 before detaching cable 12. Valve 10 is flexibly connected to wire rings 11 via loops 14. Loops 14 can be made of the same elastomeric material as valve 10. It is desired for valve 10 to be free to slide on wire rings 11 in order to “seat” itself in the best position. A single ridge 17 or multiple ridges can be added for a better hemostatic seal.

Referring now to FIG. 3, flexible cable 12 can be a stranded stainless steel cable of 2-3 mm diameter with a threaded tip engaging threaded coupler 13. An M2 or M3 thread is suitable. Wire rings 11 can be made of a continuous piece of separate rings. They can also be made in a mesh configuration. The wire can be stainless steel (such as type 316 full hard), Nitinol, beryllium copper or a polymeric material. In the preferred embodiment the diameter of wires 11 is about 0.2-0.5 mm for stainless steel wires and 0.3-6 mm for Nitinol wires, exact diameter depends on number of wire loops used. The number of wire loops can be from 1 to 10 and preferably from 2 to 5. Wires 11 can also be bent serpentine-style, as shown in FIG. 3, or have a polymeric support pad to spread the load over the top of the atrium. The load is caused by the ventricular blood pressure during valve closure.

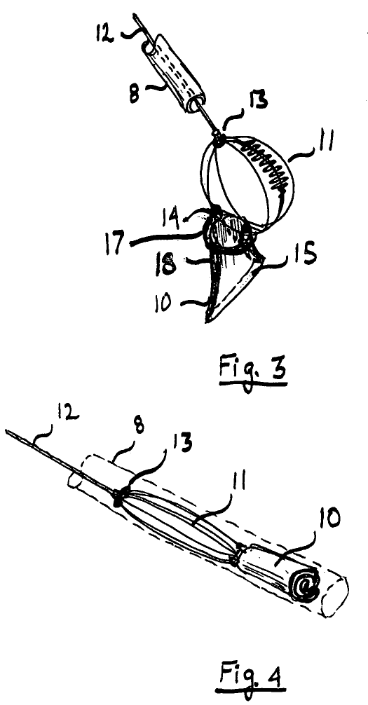

Valve 10 is a short (typically 15-40 mm long) piece of tubing of a diameter selected to fit the mitral valve. Different diameters may be needed for different size of valves. The wall thickness is around 1 mm but can be as thin as 0.3 mm. In the relaxed position the bottom part is formed to stay closed along a straight line 15, forming a valve. To help keep the shape of the valve and resists valve prolapse at high blood pressures, stiffening ridges 18 are added at both edges. The elastomeric material used for valve 12 can be synthetic, such as polyurethane or silicone rubber, or can be animal based such as pericardium. It can also be artificial or actual human tissue, even tissue grown from valve recipient, using novel methods recently developed for rapidly growing tissues on a support structure. The valve and wires can be coated with any of the well known beneficial coatings such as hydrophobic, anti-clotting, anti-inflammatory or any drug eluting coating. FIG. 4 shows a possible way of rolling up valve 10 and compressing wires 11 to fit into catheter 8. Note that after deployment valve 10 will slide on wires 11 for best seating position. This minimizes the transmission of forces to adjacent tissue and in particular the aortic valve. The present design will fit into a size 28 Fr (about 9 mm) catheter, and even in smaller sizes if valve is made of elastomeric material thinner than 1 mm.

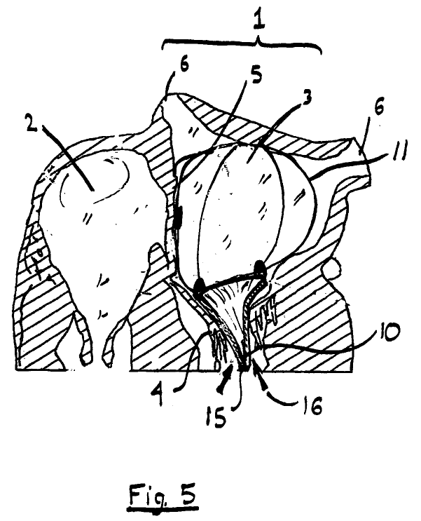

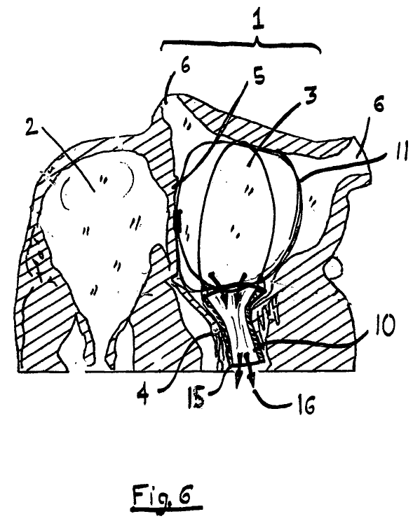

FIG. 5 shows the valve in the closed position, where blood pressure 16 keeps bottom linear seal 15 closed.

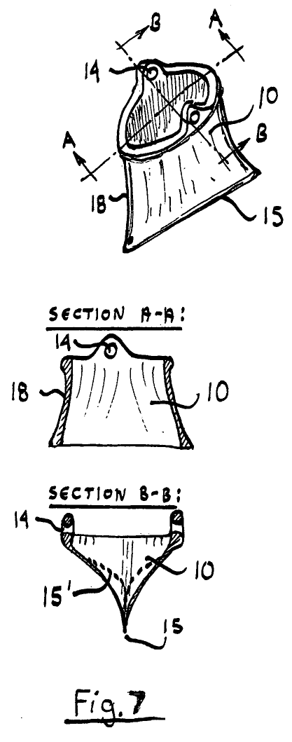

FIG. 6 shows the valve in the open position, where blood pressure 16 from the left atrium opens the seal 15 and deforms valve into a more circular shape.

It is important to make linear seal 15 very light, as wall of valve 10 needs to be very soft and flexible, in order to minimize pressure drop across valve. A sealing pressure between zero and 5 grams is sufficient, as the large pressure during ventricular contraction forms the seal.

FIG. 7 shows two cross section of the elastomeric valve. Lips forming linear seal 15 are significantly thinner than stiffening ribs 18 at two ends of valve. Under pressure the area of the linear seal increases, as shown by dotted line 15′. Stiffening ribs 18 can be further reinforced by an embedded wire.

Valve 10 can also be shaped to have a snap action, opening wider by itself once opened somewhat by the flow of blood.

While the preferred embodiment describes a mitral valve it is clear that the same invention can also be used for replacing the tricuspid valve, with the support structure deployed in the right atrium.

It is expected that over time the artificial valve will become permanently attached to the mitral valve annulus by formation of scar tissue and other well known mechanisms. Such attachment can be promoted by a suitable texture on the outside of the valve. It is known that a velour-like texture generates particularly strong bonds. When such bonding is relied on, the support rings or mesh can be made from a bio-absorbable material similar to the materials used in absorbable sutures. By the time the support structure dissolves, the artificial valve is permanently attached to the annulus of the natural valve.

What is claimed is:

1. A method of installing an artificial mitral valve, method comprising of:
introducing an artificial valve attached to a support structure into the left atrium;
deploying the support structure to fill most of the left atrium; and
supporting said valve from said support structure.

2. A method of installing an artificial tricuspid valve, method comprising of:
introducing an artificial valve attached to a support structure into the right atrium;
deploying the support structure to fill most of the right atrium; and
supporting said valve from said support structure.

3. An artificial cardiac valve comprising of a support structure and a valve, said valve is shaped as a short elastomeric tube having one end substantially round at the other end substantially linear.

4. An artificial valve as in claim 3 wherein said support structure, when deployed, is larger than said valve.

5. An artificial valve as in claims 1,2 or 3 wherein at least parts of said valve are made of one of the following materials: silicone rubber, polyurethane, animal tissue, human tissue and artificial human tissue.

6. An artificial valve as in claims 1, 2 or 3 wherein said valve incorporates snap action.

7. An artificial valve as in claims 1, 2 or 3 wherein said support structure is made of absorbable polymer.

8. An artificial valve as in claims 1, 2 or 3 wherein said support structure is made of flexible wire rings.

9. An artificial valve as in claims 1, 2 or 3 wherein said support structure is made of a mesh.

10. An artificial valve as in claims 1, 2 or 3 wherein said valve can slide on said support structure.

11. An artificial valve as in claims 1 or 2 wherein said method is performed percutaneously.
